(19) United States
(12) Patent Application Publication
(10) Pub. No.: US 2005/0221418 A1
(43) Pub. Date: Oct. 6, 2005

(54) Enhanced bacterial endospore detection method and system

(75) Inventors: Nicholas Francis Fell JR., Jessup, MD (US); Paul Matthew Pellegrino, Columbia, MD (US); James Bryce Gillespie, Ellicott City, MD (US)

Correspondence Address: U S ARMY RESEARCH LABORATORY;ATTN AMSRL CS CC IP, 2800 POWDER MILL RD, ADELPHI, MD, 207831197, US

(73) Assignee: U.S. Army Research Laboratory, Adelphi, MD (US)

(21) Appl. No.: 10/828,520

(22) Filed: Apr. 6, 2004

(51) Int. Cl. C12Q 1/04

(52) U.S. Cl. 435/34

(57) ABSTRACT

A method and system is presented for the detection of bacterial endospores down to limits of less than 500 CFU/mL. The method is based on the presence of a marker compound in bacterial endospores, dipicolinic acid (dpa). When complexed with Tb and excited in the UV range or by a laser source, the dpa enhances the photoluminescence emission of Tb by several orders of magnitude. A method is presented that eliminates interference from other biological materials and chemicals, thereby permitting only bacterial endospores to result in a positive response. The presence of phosphate or organophosphate ions will reduces the observability of detection. Accordingly, the present invention overcomes this problem through the addition of AlCl3. The present invention provides methods for enhanced the release of dpa, involving both mechanical and chemical methods, which results in at least a 200-fold increase in dpa release over the prior art.

### BACKGROUND OF INVENTION

Technical Field

The present invention relates to the detection of bacterial endospores for the further protection of humans from disease and deleterious health consequences. Accordingly, the military and homeland defense communities, along with the pharmaceutical industry will find interest in the present invention. The present invention is also of interest to the medical community for ensuring sterility in operating suites, food processing and preparation industry for prevention of food poisoning, and treaty verification, waste/water treatment.

The detection of bacterial endospores is a significant challenge in bioanalytical chemistry. The endospore is a dormant stage in the life cycle of some members of the genera Bacillus and Clostridium. A number of these species can cause disease, food poisoning, or food spoilage, so their detection is important in both commercial and medical sectors. The method we describe in this paper is intended for first-alert use. In this intended deployment the method is used to monitor the background and when the signal level increases significantly, an alert is issued to trigger other more specific and time-consuming techniques.

The bacterial endospore is mostly aptly described as a dormant state in the life cycle of certain genera of bacteria (e.g. Clostridium and Bacillus). As depicted in FIG. 1. The bacterium develops this dormant form (endospore) when there is a lack of nutrients or when it is exposed to some other adverse condition that could threaten the cell. In the process of forming the endospore, the vegetative bacterium segregates all the necessary components to restart its life cycle into a small region inside itself called the forespore.

This figure graphically depicts the process of sporulation (the transition from active, vegetative bacterium to inactive, spore form of the bacterium). The cell 100 initially has its DNA 101 “floating” in the cytoplasm 102 that is contained by the cytoplasmic membrane 103. The outermost layer of the cell is the cell wall 104. When the cell 100 experiences stress 110, such as the lack of food or extreme environmental conditions, it will begin to form a spore. The first step in this process is to isolate the DNA 101 in a region of the changing cell called the forespore 111. The spore cell wall 120 then forms around the DNA 101. Water is ejected from the region within the spore cell wall 120 and the layers of protective organic material begin to build up around the DNA 101, i.e., spore coat formation 130. Once the spore has completely formed, the mother cell 140 usually ruptures liberating the spore 150 into the environment.

Thus, FIG. 1 gives a pictorial view of the process of sporulation. In other words, the endospore continues to form by expelling most of the water from the interior and by surrounding the forespore with several resistant coatings (cortex, inner coat and outer coat). In most species, the final step is the lysis or destruction of the mother cell 140 that formed the endospore. The final product of this process, the liberated bacterial endospore 150 is a rugged package that contains the all the necessary components to reform the vegetative bacterium.

Endospores are one of the hardiest forms of life in nature resisting desiccation, UV expose, boiling and most anti-microbial compounds. An example of longevity was demonstrated recently when a bacterial endospore that had been encased in amber for 50 million years was recovered and grown to form a colony of active bacteria. This invention disclosure will describe a disruption process, when coupled with a recently develop endospore detection technique produces a highly sensitive endospore detection capability that possess several necessary advantages over its predecessor.

The technique mentioned above is the detection of bacterial endospores by terbium-dipicolinate photoluminescence was described in U.S. Pat. No. 5,876,960. This technique uses a marker chemical, e.g., dipicolinic acid (hereinafter DPA) present in endospores and complexes it with a lanthanide (terbium or europium) and upon excitation with UV light (˜280 nm) this compound emits light in bands at 490,545,590,620 nm for the terbium complexes. Since DPA only occurs naturally in bacterial endospores, and the luminescent properties depend on a unique energy transfer mechanism of the terbium-DPA complex this technique provides unambiguous detection of bacterial endospores. Addition of Tb³⁺ to the aqueous solution will result in complexation with the dpa²⁻ released from the spore. When irradiated with UV light at the dpa absorption maximum, [Tb(dpa)ₙ]³⁻²ⁿ exhibits a greatly enhanced luminescence compared to Tb³⁺ alone. The observed luminescence is shifted far from the excitation wavelength and is narrower than normal molecular luminescence signatures. The long Stoke's shift of the emitted light also provides discrimination from naturally occurring biological fluorescence.

Therefore, the detection of the Tb³⁺ luminescence signature is a unique marker for the presence of dpa and thus bacterial endospores. In our experiments we use an excess of Tb³⁺, so the form of the complex is [Tb(dpa)]⁺. Previous research has determined that energy transfer from the ligand (dpa²⁻) to the terbium excited states leads to this enhancement.

Studies indicate that the technique will only react in a positive sense to raw DPA or DPA obtained from bacterial endospores in solution. The technique also is immune to most interfering compounds, except phosphate, due to the high affinity of terbium for DPA in solution.

Up to this point most of the activity surrounding this technique utilized bacterial endospores that were untreated in terms of extracting DPA from the spore. These studies relied on the small fraction of DPA that was available from the endospores when they were placed in aqueous buffered solutions. Endospores can contain anywhere from 2-15% DPA in weight percentage. Examination of the literature and our experimental studies indicated that only a fraction (˜ 1/10) of the DPA was accessible for use in the detection scheme when no active extraction techniques were used (i.e. the sample was just placed in an aqueous solution). Other studies indicated that exhaustive washing procedures could remove all DPA that was normally available from just suspending sample in an aqueous environment thus defeating the technique without active extraction. The endospores still contains ample DPA, but the remaining DPA is inaccessible due to the fact it is intercalated in the endospore coatings and the core. Therefore, the prior art shortcomings associated with the terbium-DPA sensing technique illustrate the need for improved extraction techniques.

Prior art commercial fluorescence methods have had detection limits no less than 100,000 CFU/mL.

The prior art reports that phosphate compounds, such as a buffer solution, has an inhibiting effect on the photoluminescent emission from terbium dipicolinate. Two explanations are possible for this observation. The first is that the phosphate anion has a high affinity for Tb⁺³ and may displace dpa from it. Secondly, the phosphate anion may quench the excited dpa molecules before they transfer their energy to the Tb⁺³.

It has been reported that Al⁺³ is used in water processing facilities to remove phosphate ions.

Therefore, there exists a need to minimize and/or eliminate the deleterious effects of phosphate containing compounds.

### SUMMARY OF INVENTION

The present invention is devised in view of the problems of prior art, and its purpose is to provide a method that enhances the detection of bacterial endospores. This is accomplished by the use of Tb photoluminescence.

The first objective of the present invention is to enhance the detection of bacterial endospores. This includes the identification and analysis of various chemical and biological materials for interference in bacterial endospore detection. This objective includes preventing both false positive and false negative indications of presence of bacterial endospores. This objective prevents false positive signals for samples that do not contain bacterial endospores. This objective also prevents false negative identification, which results from the reduction or elimination of positive signals from samples containing bacterial endospores.

A second objective of the invention is to utilize marker chemicals and marker chemical enhancement agent to reduce the deleterious effect of interferants to bacterial endospore detection.

A third objective of the invention was to improve the limit of detection by increasing the amount of marker chemical extracted from the bacterial endospores by several mechanical and chemical processes for extracting additional marker chemical.

A fourth objective of the invention is to provide a release agent for the extraction of substantially all of the available marker chemical.

A fifth object of the invention is to provide a method of extracting substantially all of the available DPA in less than 10 minutes.

A sixth object of the invention is to provide an optical system method utilizing a laser.

A seventh object of the invention is to provide a detection system and method with a limit of detection of lower than 500 CFU/ml.

### BRIEF DESCRIPTION OF DRAWINGS

In the drawings:

FIG. 1 illustrates the process of sporulation in bacterial endospores;

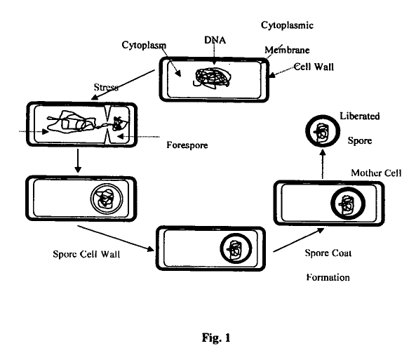

FIG. 2 illustrates several positive responses to the presence of various bacterial endospores in an embodiment of the present invention;

FIG. 3 illustrates the effect of phosphate on a positive response and the mitigating effect due to the addition of several types of salt solution in an embodiment of the present invention;

FIG. 4 illustrates a linear graph of a limit of detection for bacterial endospores in an embodiment of the present invention;

FIG. 5 illustrates an embodiment of the present invention bacterial detection system;

FIG. 6 illustrates an embodiment of the present invention's sample flow device; and

FIG. 7 illustrates an embodiment of the present invention's optical analysis device.

### DETAILED DESCRIPTION

The following section describes embodiments of the present invention based on the figures.

Under the present invention, the samples in each group of experiments were brought to a constant volume by the inclusion of a buffer solution, wherein said level of Tb and B. globigii were held constant within each set to permit proper comparisons. All of the samples were made in aqueous Trizma buffer at pH 7.6. Three replicate measurements were made on each sample and the average value for the three replicates was used for succeeding calculations and in all plots. The signal from each set of samples was normalized to a standard with the same bacterial endospore concentration and total volume.

Under the present invention, three classes of materials were examined to determine their effect on bacterial endospore detection. Nine organic chemicals, seven inorganic salts, and fourteen biological materials were examined for both false positive and false negative responses. A false positive occurs when a material other than a bacterial endospore give a signal above the Tb background. None of the materials examined generated a false positive signal.

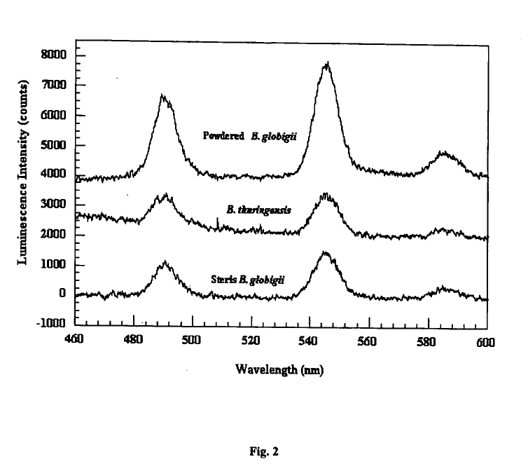

Based upon the present invention, only bacterial endospores yielded positive responses, as seen in FIG. 2. FIG. 2 includes the responses for two samples of B. globigii and a commercial insecticide containing 0.8% B. thuringensis. The Steris B. globigii was washed by centrifugation, yet a signal was still detected from the dpa remaining in the spores. FIG. 2 illustrates the positive responses from bacterial endospores. Top-powdered B. globigii 6.165×10⁸ CFU/mL, intensity offset by +4000; Middle-Thuricide®, containing 0.8% B. thuringensis, diluted by a factor of 40, intensity offset by +2000; Bottom-Steris B. globigii 1.3×1 0⁹ CFU/mL.

Several of the materials in the test set did result in reductions in the signals observed from a fixed amount of B. globigii. Significant degradation of signal detection occurred with addition of phosphate-containing chemicals. K₂ HPO₄, for example, had the most deleterious effect the signals. These results demonstrated the need for a method to deal with phosphate interference.

The present invention reduces and/or eliminates the deleterious effect of phosphates. Samples of B. globigii experienced a decrease in signal of up to 15% when exposed to 8.5×10⁻⁵ M K₂ HPO₄. At the same concentration of K₂ HPO₄, dpa itself had a 65% decrease in signal detection strength.

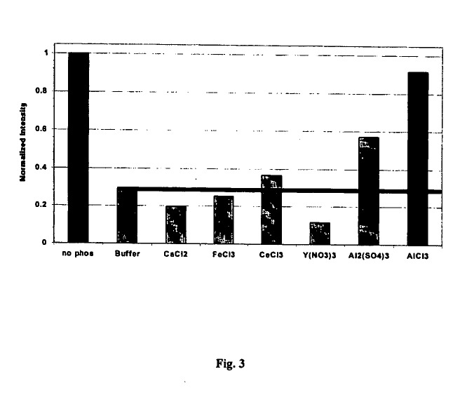

The present invention provides various inorganic salts to reduce the interference of phosphates. FIG. 3 illustrates signal recovery on the addition of salt solutions to samples containing bacterial endospores. The signal was normalized to the intensity (no phosphate) of the Tb/dpa stock with buffer added to compensate for the volume of salt solution added. Buffer represents the normalized signal level of the Tb/dpa/phosphate stock with buffer added to compensate for the volume of salt solution added. The beneficial effects of Al₂(SO₄)₃ and AlCl₃ are evident.

In an embodiment of the present invention, salts with cations were utilized. The prior art reports the use of Al⁺³ in water processing facilities to remove phosphate ions. The utility of the present invention's approach is evident from these results. The salts with anions containing a negatively charged oxygen were less effective than those without a negatively charged oxygen and served to reduce the signal or hinder the beneficial effects of the cation. This is a direct result of the high affinity of Tb⁺³ for negatively charged oxygens. When AlCl₃ was added to samples containing dpa or Dugway B. globigii, the signal level was reduced by up to 15% for dpa and maintained for the Dugway B. globigii on exposure to 8.5×10⁻⁵ M K₂ HPO₄, provided that the dpa was added before or at the same time as the phosphate. This graphically shows the beneficial effect of addition of AlCl₃.

The present invention provides an efficient and effective method of enhancing the release of dpa from the bacterial endospores. It should be noted that the prior art states that less than 10% of the available dpa is actually released by suspension in aqueous buffers. In an embodiment of the present invention, washed commercial suspension of B. globigii was utilized that had a significantly lower level of readily available dpa upon suspension in aqueous buffer other B. globigii. This was performed as a means of ensuring the robustness of the present invention's mechanical and chemical processes of extracting increasing amounts of dpa from the endospores.

The present invention's extraction methods include pressure boiling; mechanical agitation/abrasion, surfactants, and germination solutions are used.

Table I summarizes various dpa release methods of the present invention. The maximum enhancement is the ratio of the maximum signal achieved and the room temperature standard with the same concentration of bacterial endospores. The maximum enhancement has been normalized to the level of dpa release achieved from boiling. The normal amount of dpa released by placing the spores in water is 5-10%. The 112% listed for DDA treatment is within the reproducibility of the experiment and biological diversity.

The mechanical means of the present invention include sonication, sonication with glass beads, e.g., 40 nm and 200 nm, agitation, e.g., shaking, with glass bead and diatomaceous earth, and heating. Sonication and shaking with glass beads crushes the endospores between the glass beads. Shaking with diatomaceous earth abrades the endospores on the sharp facets of the diatomaceous earth. Agitation with glass beads or diatomaceous soil increases the extracted dpa to 34% and 26% of the total dpa available in the endospore, respectively.

The present invention utilizes three chemical means of extracting more dpa. The first mixes a solution of cetyltrimethylammonium bromide (CTAB) with a sample of the bacterial endospore suspension and heat the mixture to 50° C. Samples are then removed at predetermined time intervals, mixed with Tb solution, and the photoluminescence emission collected. These process steps take approximately two hours to reach the maximum release and increased the dpa to 40% of the total dpa available in the endospore.

The second chemical method induces the endospores into germinating, since the first step in that process is the expulsion of all of the dpa from the endospore. This is accomplished by adding a mixture of alanine, asparagine, and glucose to a sample of the endospore suspension, sampling at intervals, and collecting the photoluminescence emission. This method released 32% of the available dpa in the endospore and took approximately three hours. In addition, pre-heating endospores achieves almost complete release of dpa from the spores with either alanine alone or the alanine-asparagine-glucose mixture. However, it still requires approximately 2 to 3 hours to reach the maximum level of dpa release.

The third chemical method involves the addition of a marker chemical release agent, which facilitates the extraction/release of the marker chemical, to the endospore suspensions. This is performed in a similar manner to the CTAB addition. In an embodiment of the present invention, dodecylamine (hereinafter “DDA”) was added to a sample suspension to enhance the release substantially all of the dpa, as determined by boiling for example, from the bacterial endospores in less than a few minutes. The dpa release with DDA is a strong function of relative endospore-to-DDA concentration and temperature. Once a threshold level of DDA is reached, the rate of dpa release is independent of endospore-to-DDA ratio.

Accordingly, DDA addition is the preferred embodiment of the present invention for releasing dpa from bacterial endospores.

The rate also increases as a function of temperature. DDA was added to a sample suspension to enhance the release of substantially all of the dpa from the bacterial endospores in approximately 2 minutes at 80° C. However without the DDA additive, the rate of increase is substantially slower even with upon increased temperature, as can be seen in Table 1 with an enhanced release of dpa to 100% in 15 minutes at 100° C. Thus, the preferred embodiment of the present invention for extracting dpa from the bacterial endospores includes the addition of DDA.

The present invention permits the release of substantially all of the dpa from the endospores resulting in a highly sensitive detection of bacterial endospores. As stated earlier, the prior art has limits of detection (LOD) of greater than 100,000 CFU/mL of suspension for endospores, such as B. globigii, however, the present invention detects less than 5000 CFU/mL with a release agent. The figures indicate that detection of less than 10,000 CFU/mL of BG has been achieved using the DDA treatment with a signal-to-noise ratio of greater than 6, indicating that our LOD is less than 5000 CFU/mL. This level of detection permits detection at the 10 agent-containing particle per liter of air level in less than 10 minutes of sampling at the 500 L/minute rate and concentration to 1 mL of liquid.

The present invention permits the level of detection, with a DDA treatment to reach detection limits of less than 500 CFU/mL. In addition, the present invention has achieved detection levels of approximately 20 CFU/mL.

The results illustrate the high sensitivity of the present invention for bacterial endospore detection. The present invention permits the detection of less than 500 CFU/mL of endospores in less than 5 minutes with DDA for enhanced release of dpa from the endospores. The present invention's method is principally immune from false positive responses. Thus, through the use of a marker chemical enhancement agent, such as AlCl₃, to complex any phosphate present in a sample the invention substantially eliminates one of the most significant interferences in detection of the bacterial endospores.

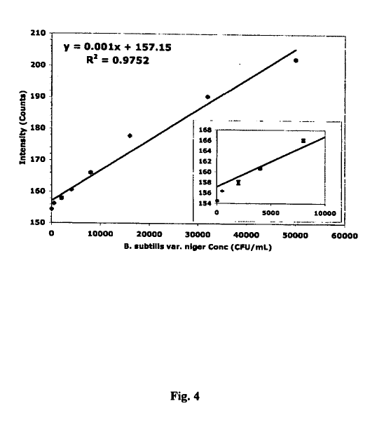

The present invention achieves a routine limit of detection of 500 CFU/mL for endospores, such as B. subtilis var. niger. This detection limit is at least a factor of 200 lower than those previously reported and are illustrated in FIG. 4.

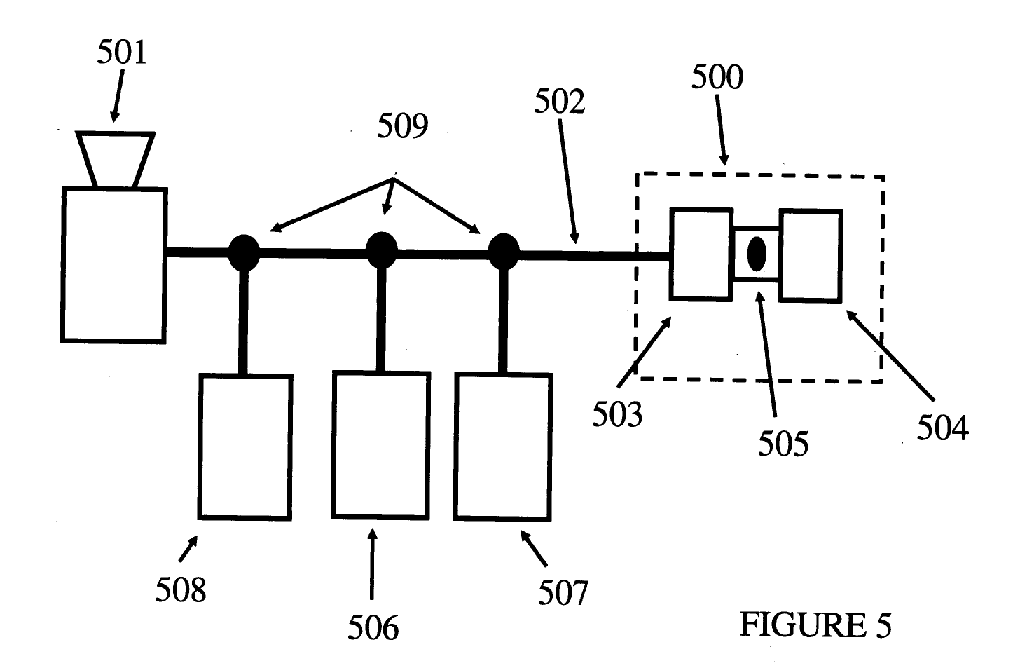

FIG. 5 illustrates an embodiment of the present invention's bacterial endospore detection system. The optical detection device 500 is coupled to a sampler 501 by a flowpath 502. The sampler 501 samples aerosols, airborne particle or liquids depending on the embodiment. The optical detection device 500 is comprised of a sample flow device 503 and a optical analysis device 504, which are both coupled to and include an optical flow cell 505. The sample flow device 503 provides a mechanical mechanism for transferring the sample from sampler 501 through the flowpath 502 to the optical flow cell 505 for bacterial endospore detection.

The optical analysis device 504 permits the detection of the presence of bacterial endospores in a sample transferred from the sampler 503 through the flowpath 502 to the optical flow cell 505. The bacterial detection system according to an embodiment of the present invention is further comprised of a marker chemical complexing agent reservoir 506 coupled to the optical detection device 500 by a flowpath 502. The embodiment further includes a marker enhancement agent reservoir 507 coupled to the optical detection device 500 by a flowpath 502. The embodiment further includes a release agent reservoir 508 coupled to the optical detection device 500 by a flowpath 502. The bacterial detection system further includes mixing zones 509 to combine the contents of the reservoirs. In another embodiment, the mixing zones 509 are heated zones, which apply heat for increasing the temperature of the various agents of the present invention.

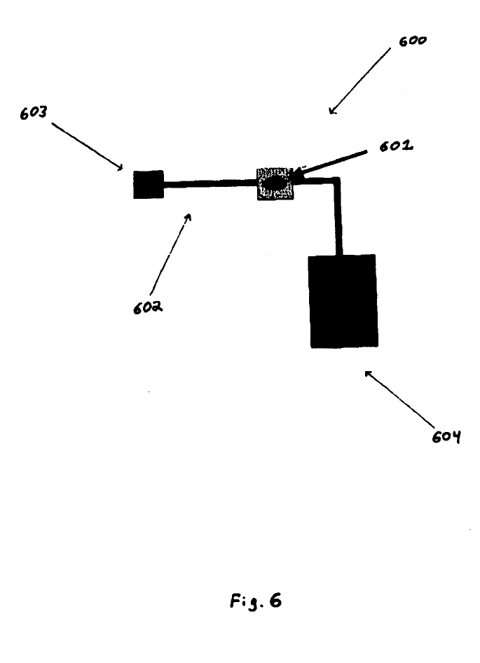

FIG. 6 illustrates an embodiment of the present invention's bacterial endospore detection system's sample flow device 600, which is coupled to an optical flow cell 601. The sample flow device 600 provides a mechanical mechanism for transferring the sample from sampler 501 through the flowpath 602 to the optical flow cell 601. A sample introduction port 603 is coupled to the optical flow cell 601 by the flowpath 602. A waste container 604 is also provided for collecting the sample subsequent to its analysis from the optical flow cell 601.

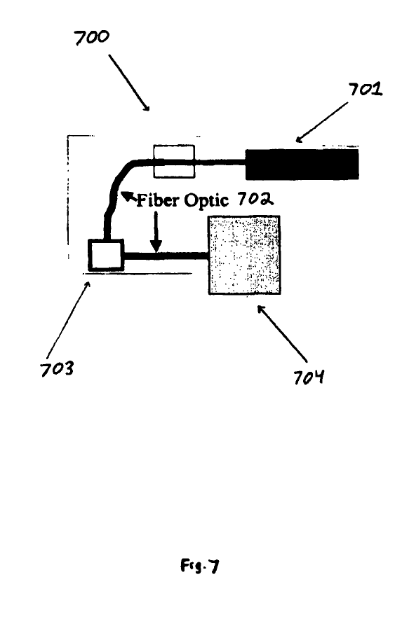

FIG. 7 illustrates an embodiment of the present invention's bacterial endospore detection system's optical analysis device 700, which is comprised of a UV laser 701, e.g., a compact, quadrupled Nd:YLF laser (262 nm), coupled to an optical fiber 702 for delivery of the UV photons to the optical flow cell. An optical fiber 702 collects the emitted visible photoluminescence from the optical flow cell 703 and delivers it to the optical analyzer 704. The optical analyzer 704, in this embodiment is a comprised of a spectrometer, however it could also be a prior art optical device, such as an optical filter and photodetector for wavelength detection.

The present invention combines the biospore detection process improvements described above and with the improved optical analysis device 700, as illustrated in FIG. 7. One of the principal improvements over the prior art is the change in the optical source from a UV lamp filtered with a monochromator to a laser 701, e.g., a compact, quadrupled Nd:YLF laser, in the embodiment of FIG. 7, emitting a beam of light at 262 nm. In addition, various optical analyzers can be used, in addition to and in place of the compact spectrometer of the embodiment illustrated in FIG. 7. The present invention achieves an improvement on the order 200 of magnitude in sensitivity for detecting bacterial endospores as compared to the prior art.

The addition of a laser 701 as the source permits greater flexibility in other components of a detection system. Since the laser provides approximately 1000 times the excitation intensity of a lamp, it permits us to use less sensitive detectors, such as the compact spectrometer. The ability to use an inexpensive spectrometer allows us to identify spectroscopic interference more easily and thus prevent false detection alerts. The laser 701 also allows simpler implementation of time-resolved luminescence to eliminate the background signal from the uncomplexed Tb³⁺ that is present in the solution.

The present invention provides the advantage of a compact bacterial endospore detection system that exhibits improved sensitivity for bacterial endospores. The present invention also provides a substantial reduction in size of the detection system compared to the prior art.

Those skilled in the art will recognize that the method of the present invention has many applications, and that the present invention is not limited to the representative examples disclosed herein. Although illustrative, the embodiments disclosed herein have a wide range of modification, change and substitution that is intended and in some instances some features of the present invention may be employed without a corresponding use of the other features.

Moreover, the scope of the present invention covers conventionally known variations and modifications to the system components described herein, as would be known by those skilled in the art. Accordingly, it is appropriate that the appended claims be construed broadly and in a manner consistent with the scope of the invention.

TABLE 1

| | Maximum | Time to | |
|---|---|---|---|
| Method | Enhancement | Max. Signal | Disadvantages |
| Glass Beads | 34% | 2 min | Agitation Required |
| | | | Settling Required |
| Diatomaceous | 26% | 12 min | Agitation Required |
| Earth | | | Settling Required |
| CTAB | 40% | 120 min | |
| Amino | 32% | 180 min | High Concentrations |
| Acid/Sugar | | | |
| DDA | 112% | 2 min @ 80° C. | |
| Boiling | 100% | 15 min | Significant |
| | | | Heating Required |

1. A method of detecting bacterial endospores comprising:
providing a sample;
providing a marker chemical complexing agent;
providing a laser;
determining if a marker chemical is present in said sample;
if said marker chemical is present, complexing said marker chemical with said marker chemical complexing agent exposing said sample to said laser; and
detecting the presence of bacterial endospores in said sample.

2. The method according to claim 1, wherein said marker chemical is dipicolinic acid.

3. The method according to claim 1, wherein said marker chemical complexing agent is a terbium containing compound.

4. The method according to claim 1, wherein said marker chemical complexing agent is heated above 30° C.

5. The method according to claim 1, further comprising providing a release agent.

6. The method according to claim 5, wherein said release agent releases substantially all of said marker material from said bacterial endospores.

7. The method according to claim 5, wherein said release agent is dodecylamine.

8. The method according to claim 5, wherein said release agent is heated above 30° C.

9. The method according to claim 1, wherein said method further comprises detecting less than 100,000 CFU/mL of endospores.

10. The method according to claim 1, wherein said method further comprises detecting less than 10,000 CFU/mL of endospores.

11. The method according to claim 1, wherein said method further comprises detecting less than 5,000 CFU/mL of endospores.

12. The method according to claim 1, wherein said method further comprises detecting less than 1,000 CFU/mL of endospores.

13. The method according to claim 1, wherein said method further comprises detecting less than 500 CFU/mL of endospores.

14. The method according to claim 1, wherein said method further comprises detecting less than 100 CFU/mL of endospores.

15. The method according to claim 1, wherein said method further comprises detecting less than 20 CFU/mL of endospores.

16. The method according to claim 1, wherein said detection of the presence of bacterial endospores occurs in less than 10 minutes.

17. The method according to claim 1, wherein said detection of the presence of bacterial endospores occurs in less than 5 minutes.

18. The method according to claim 1, wherein said detection of the presence of bacterial endospores occurs in less than 3 minutes.

19. The method according to claim 1, wherein said method further includes providing a marker chemical enhancement agent and combining said agent with said sample.

20. The method according to claim 1, wherein said marker chemical enhancement agent is an AlCl₃ containing compound.

21. The method according to claim 1, wherein said marker chemical enhancement agent is heated above 30° C.

22. The method according to claim 1, wherein said laser emits light at a wavelength between 260 and 280 nanometers.

23. The method according to claim 1, further comprising:
agitating said sample.

24. The method according to claim 23, where said agitation includes:
sonic, mechanical and heating.

25. A bacterial endospore detection system comprising:
an optical detection device;
wherein said optical detection device further comprises an optical flow cell;
flowpath;
sampler; and
marker chemical complexing agent reservoir.

26. The system according to claim 25, wherein said optical detection device includes a sample flow device.

27. The system according to claim 25, wherein said optical detection device includes an optical analysis device.

28. The system according to claim 25, further comprises a marker chemical enhancement agent reservoir.

29. The system according to claim 25, further comprises a release agent reservoir.

30. The system according to claim 25, wherein said flowpath includes at least one mixing zone.

31. The system according to claim 30, wherein at least one mixing zone is heated.
